(19) United States
(12) Patent Application Publication
(10) Pub. No.: US 2025/0160712 A1
(43) Pub. Date: May 22, 2025

(54) MENTAL WELL-BEING SOLUTION FOR DETERMINING TO IDENTIFY AT-RISK USER BASED ON A WELLBEING INDEX

(71) Applicant: MEANDMINE INCORPORATED, Mountain View, CA (US)

(72) Inventor: Wen Hsin Huang, San Jose, CA (US)

(21) Appl. No.: 18/949,938

(22) Filed: Nov. 15, 2024

Related U.S. Application Data
(60) Provisional application No. 63/599,912, filed on Nov. 16, 2023.

(51) Int. Cl. A61B 5/16; G16H 10/20; G16H 15/00

(52) U.S. Cl. CPC A61B 5/165; G16H 10/20; G16H 15/00

(57) ABSTRACT

In some implementations, a method may include obtaining, using measurement system, a first set of user data. In addition, the method may include inputting, the first set of user data into a predictive model to determine a first score. The method may include determining, using the score, an indicator associated with a mental state status of the user. Moreover, the method may include determining, based on the indicator, a category associated with a self-regulation of the mental state status of the user; determining a first gamification application for the user based on the category; and presenting, by the measurement system, the category, and the gamification application to the user.

### CROSS-REFERENCE TO RELATED APPLICATIONS

This application claims priority under 35 U.S.C. § 119 (e) to U.S. Provisional Application No. 63/599,912, filed Nov. 16, 2023. The entire content of the priority application is hereby incorporated by reference.

### TECHNICAL FIELD

Embodiments of the present disclosure relate generally to mental well-being. More particularly, embodiments of the disclosure relate to determining mental well-being of a user using a predictive model.

### BACKGROUND

Students are currently facing significant mental health challenges, with one in five students affected. Teachers find themselves increasingly overwhelmed as they strive to meet the diverse needs of each student while providing the individualized support necessary for their success. The increasing prevalence of mental health challenges among children is significantly affecting their ability to succeed in the academic environment. Conditions such as anxiety, depression, and attention-deficit/hyperactivity disorder (ADHD) often present as challenges in concentration, disruptive behaviors, and social withdrawal, which in turn impede academic performance and overall engagement in the classroom. These difficulties can result in heightened absenteeism, diminished grades, and strained relationships with peers and educators. For example, the rising rates of depression are contributing to an additional 26 days of school absenteeism. Moreover, the stigma associated with mental health issues frequently discourages children from seeking the support they require, thereby intensifying the problem and obstructing their academic advancement.

Schools are instrumental in the early identification and resolution of these matters. Access to mental health resources and early intervention is critical for assisting children in managing their symptoms and developing effective coping mechanisms. By cultivating a supportive and empathetic school environment, educators can establish a setting where children feel secure in discussing their challenges and obtaining the necessary assistance. A collaborative effort among schools, families, and mental health professionals is essential to ensure that children facing mental health issues could achieve their full academic and social potential.

Current market solutions lack the ability to provide real-time monitoring and tracking of students' daily mental well-being, as well as actionable insights for teachers, therapists, and school administrators. This gap limits the capacity to deliver timely support and interventions, resulting in missed opportunities for early help and comprehensive care.

Student mental health issues are on the rise, yet current approaches often lack continuous monitoring and tracking, leading to delayed interventions, inconsistent care, and increased incidents. This also limits actionable insights for educators. There is a critical need for an evidence-based, technology-driven solution that facilitates timely intervention, enhances student engagement, and reduces administrative burdens, thereby improving overall well-being, reducing staff turnover, and increasing the quality of care.

### BRIEF DESCRIPTION OF THE DRAWINGS

Embodiments of the disclosure are illustrated by way of example and not limitation in the figures of the accompanying drawings in which like references indicate similar elements.

FIG. 1 is a block diagram illustrating a system environment for a predictive system according to one embodiment.

FIG. 2 is a block diagram illustrating an example of an architecture of a predictive system according to one embodiment.

FIG. 3 is an example application that can be used to obtain a mental health status of a student according to one embodiment.

FIG. 4 is an example educator dashboard that can be used to monitor mental health status of a student according to one embodiment.

FIG. 5 is a block diagram illustrating an example of an architecture of a predictive system according to one embodiment.

FIG. 6 is a block diagram illustrating an example of personalized self-regulation games according to one embodiment.

FIG. 7 is a block diagram illustrating an example of a predictive mental health flagging system according to one embodiment.

FIG. 8 is a flowchart illustrating a process of identifying at-risk students according to one embodiment.

FIG. 9 is a flowchart illustrating a process of identifying a mental status of students according to one embodiment.

FIG. 10 is a flowchart illustrating a process of identifying a mental status of students according to one embodiment.

### DETAILED DESCRIPTION

Various embodiments and aspects of the disclosures will be described with reference to details discussed below, and the accompanying drawings will illustrate the various embodiments. The following description and drawings are illustrative of the disclosure and are not to be construed as limiting the disclosure. Numerous specific details are described to provide a thorough understanding of various embodiments of the present disclosure. However, in certain instances, well-known or conventional details are not described in order to provide a concise discussion of embodiments of the present disclosures.

Reference in the specification to “one embodiment” or “an embodiment” means that a particular feature, structure, or characteristic described in conjunction with the embodiment can be included in at least one embodiment of the disclosure. The appearances of the phrase “in one embodiment” in various places in the specification do not necessarily all refer to the same embodiment.

The system provides a personalized and engaging approach to mental well-being. Users are engaged with self-regulation, games, practices, stories, books, and activities that make learning about emotions fun and interactive.

The system includes an application for the users (e.g., students) to check in with their feelings, energy level, physical health, and social interactions. The system determines which regulation category associated with the users and the system determines personalized games, helping the users to center themselves and get ready to learn. The daily quest is assigned to users where they team up to cultivate self-regulation skills and hatch 25 emotion buddies by progressing through challenges as users play. The predictive model of the system analyzes over 160 data points to identify users who may need extra support. The system also includes an educator/clinician dashboard (“dashboard”) that displays a comprehensive view of each user's well-being, tracking the user's progress over time at schools. The dashboard provides insights into the users' emotional state, fostering a supportive environment with shared vocabulary and mindfulness practices. The dashboard utilizes longitudinal and daily well-being data of the user to improve the predictive model in identifying psychological risks among students. The system determines at-risk users ensuring the users can receive the help they need earlier.

The system empowers district schools, regional health centers and state leaders with data-driven insights to make informed decisions and proactively address student mental health risks. Using multiple data signals and machine learning, it provides real-time student screening and high-fidelity insights, supporting tier 1 and tier 2 prevention strategies to mitigate mental health issues early on.

The system provides teachers with an engaging learning tool to promote classroom emotional wellbeing. The system provides counselors/psychologist with a data-driven system for real time monitoring and AI-flagging. The system provides school districts with spot risk patterns for preventive measures and optimize resource deployment.

According to some embodiments, the method includes obtaining, using measurement system, a first set of user data. The method also includes inputting, the first set of user data into a predictive model to determine a first score. The method additionally includes determining, using the score, an indicator associated with a mental state status of the user. Further, the method includes determining, based on the indicator, a category associated with a self-regulation of the mental state status of the user. In addition, the method includes determining a first gamification application for the user based on the category. Further, the method includes presenting, by the measurement system, the category, and the gamification application to the user.

According to some embodiments, a non-transitory machine-readable medium having instructions stored therein, which when executed by a processor, cause the processor to perform operations. The operations include obtaining, using measurement system, a first set of user data. The operations also include inputting, the first set of user data into a predictive model to determine a first score. The method additionally includes determining, using the score, an indicator associated with a mental state status of the user. Further, the operations include determining, based on the indicator, a category associated with a self-regulation of the mental state status of the user. In addition, the operations include determining a first gamification application for the user based on the category. Further, the operations include presenting, by the measurement system, the category, and the gamification application to the user.

According to some embodiments, a system comprises a processor, and a memory coupled to the processor to store instructions, which when executed by the processor, cause the processor to perform operations. The operations include obtaining, using measurement system, a first set of user data. The operations also include inputting, the first set of user data into a predictive model to determine a first score. The method additionally includes determining, using the score, an indicator associated with a mental state status of the user. Further, the operations include determining, based on the indicator, a category associated with a self-regulation of the mental state status of the user. In addition, the operations include determining a first gamification application for the user based on the category. Further, the operations include presenting, by the measurement system, the category, and the gamification application to the user.

According to some embodiments, the method includes obtaining, using a first measurement system, a first data. The method also includes inputting, the first set of user data into a predictive model to determine a first score. The method further includes determining, using the score, an indicator associated with a mental state status of the user. Additionally, the method includes obtaining, using a second measurement system, a second data. The method further includes obtaining, using a third measurement system, a third data. The method additionally includes determining, using the predictive model, a wellbeing index to identify at-risk user. The method further includes sending, to a provider, a notification if the user is the at-risk user.

According to some embodiments, a non-transitory machine-readable medium having instructions stored therein, which when executed by a processor, cause the processor to perform operations. The operations include obtaining, using a first measurement system, a first data. The operations also include inputting, the first set of user data into a predictive model to determine a first score. The operations further include determining, using the score, an indicator associated with a mental state status of the user. Additionally, the operations include obtaining, using a second measurement system, a second data. The operations further include obtaining, using a third measurement system, a third data. The operations additionally include determining, using the predictive model, a wellbeing index to identify at-risk user. The operations further include sending, to a provider, a notification if the user is the at-risk user.

According to some embodiments, a system comprises a processor, and a memory coupled to the processor to store instructions, which when executed by the processor, cause the processor to perform operations. The operations include obtaining, using a first measurement system, a first data. The operations also include inputting, the first set of user data into a predictive model to determine a first score. The operations further include determining, using the score, an indicator associated with a mental state status of the user. Additionally, the operations include obtaining, using a second measurement system, a second data. The operations further include obtaining, using a third measurement system, a third data. The operations additionally include determining, using the predictive model, a wellbeing index to identify at-risk user. The operations further include sending, to a provider, a notification if the user is the at-risk user.

According to some embodiments, the method further includes obtaining, using a first measurement system, a first data. The method also includes obtaining, using a second measurement system, a second data. The method includes obtaining, using a third measurement system, a third data. In addition, the method includes inputting, the first data, the second data, and the third data into a predictive model to determine a score. Further, the method includes predicting, using the predictive model and based on the score, the mental state status of a user. The method further includes displaying, by the first measurement system, the mental state status of the user.

According to some embodiments, a non-transitory machine-readable medium having instructions stored therein, which when executed by a processor, cause the processor to perform operations. The operations further include obtaining, using a first measurement system, a first data. The operations also include obtaining, using a second measurement system, a second data. The operations include obtaining, using a third measurement system, a third data. In addition, the method includes inputting, the first data, the second data, and the third data into a predictive model to determine a score. Further, the operations include predicting, using the predictive model and based on the score, the mental state status of a user. The operations further include displaying, by the first measurement system, the mental state status of the user.

According to some embodiments, a system comprises a processor, and a memory coupled to the processor to store instructions, which when executed by the processor, cause the processor to perform operations. The operations further include obtaining, using a first measurement system, a first data. The operations also include obtaining, using a second measurement system, a second data. The operations include obtaining, using a third measurement system, a third data. In addition, the method includes inputting, the first data, the second data, and the third data into a predictive model to determine a score. Further, the operations include predicting, using the predictive model and based on the score, the mental state status of a user. The operations further include displaying, by the first measurement system, the mental state status of the user.

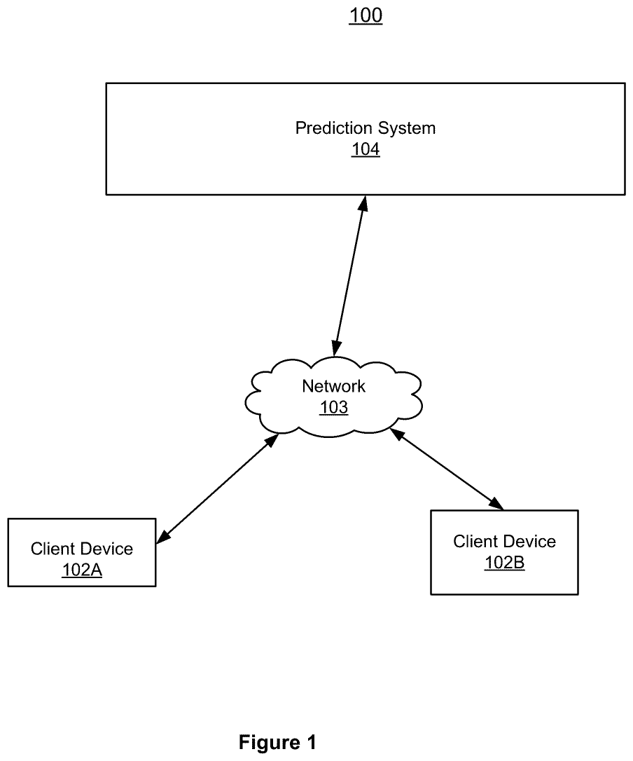

FIG. 1 presents a block diagram illustrating an environment 100 for a mental health prediction system, as per an embodiment. The environment 100 may consist of one or more client devices 102A, 102B, a network 103, and the mental health prediction system 104. The various client devices 102A and 102B may collectively be referred to as a single client device 102. In alternative arrangements, the environment 100 may incorporate different and/or additional components.

The client device 102 is a computing device capable of receiving user input as well as communicating via the network 103. While a single client device 102 is illustrated in FIG. 1, in practice many client devices 102 may communicate with the systems in environment 100. In one embodiment, a client device 102 is a computer system, such as a desktop, or laptop computer. Alternatively, a client device 102 may be a device having computer functionality, such as a personal digital assistant (PDA), a mobile telephone, a smartphone, or another suitable device. A client device 102 is configured to communicate via the network 103. In one embodiment, a client device 102 executes an application allowing a user of the client device 102 to interact with the mental health prediction system 104. For example, a client device 102 executes a browser application to enable interaction between the client device 102 and the mental health prediction system 104 via the network 103. In another embodiment, the client device 102 interacts with the mental health prediction system 104 through an application programming interface (API) running on a native operating system of the client device 102, such as IOS® or ANDROID™.

The client device 102 is a computing device capable of receiving user input and facilitating communication through the network 103. Although a single client device 102 is depicted in FIG. 1, it is recognized that multiple client devices 102 may interact with the systems within the environment 100. In one embodiment, a client device 102 may be a computer system, such as a desktop or laptop computer. Alternatively, a client device 102 might encompass devices with computing functionality, including personal digital assistants (PDAs), mobile telephones, smartphones, or other appropriate devices. A client device 102 is designed to communicate via the network 103. In one instance, a client device 102 operates an application that enables user interaction with the mental health prediction system 104. For example, a client device 102 may run a browser application that allows for interaction with the mental health prediction system 104 through the network 103. In another embodiment, the client device 102 connects with the mental health prediction system 104 via an application programming interface (API) that operates on the native operating system of the client device 102, such as iOS® or Android™.

The client devices 102 are designed to communicate through the network 103, which may encompass various local area and/or wide area networks, utilizing both wired and/or wireless communication systems. In one embodiment, the network 103 employs standard communication technologies and/or protocols. For instance, the network 103 may include communication links that utilize technologies such as Ethernet, 802.11, worldwide interoperability for microwave access (WiMAX), 3G, 4G, code division multiple access (CDMA), and digital subscriber line (DSL). Networking protocols used for communications over the network 103 include multiprotocol label switching (MPLS), transmission control protocol/Internet protocol (TCP/IP), hypertext transport protocol (HTTP), simple mail transfer protocol (SMTP), and file transfer protocol (FTP). The data exchanged over the network 103 may be formatted using various suitable formats, such as hypertext markup language (HTML) or extensible markup language (XML). In certain embodiments, some or all communication links within the network 103 may be secured using appropriate encryption techniques.

The client devices 102 provide user data to the mental health prediction system 104 and launches personalized self-regulation games that help student center themselves and get ready to learn. User data may include daily emotion data, students' in-game behavior records, reward histories, and insightful feedback from educators.

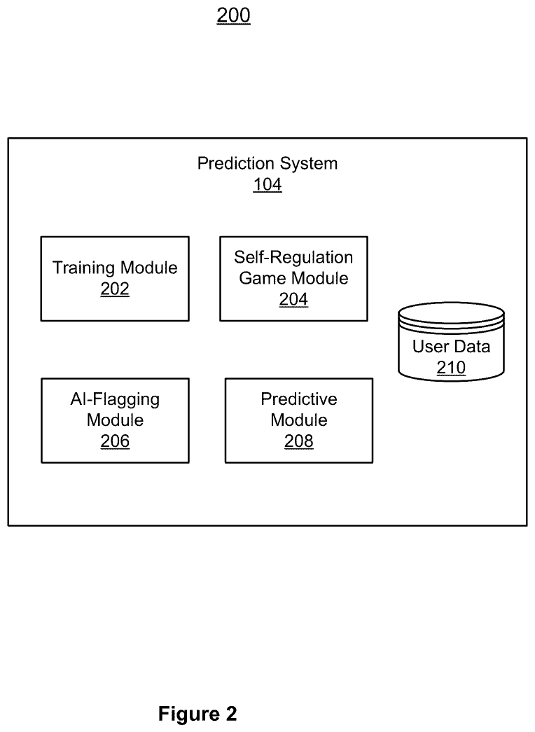

FIG. 2 is a block diagram illustrating an example of an architecture of a predictive system according to one embodiment. The predictive system 104 shown in FIG. 2 includes a training module 202, self-regulation game module 204, AI-flagging module 206, and predictive module 208. In various implementations, the predictive system 104 may incorporate additional, fewer, or alternative components tailored for specific applications. To maintain clarity regarding the system architecture, conventional components such as network interfaces, security functions, load balancers, failover servers, management, and network operations consoles are not depicted.

The training module 202 is configured to train AI models that suggest personalized self-regulation games, identify at-risk students, predict psychological risks. In this specific embodiment, which is referenced throughout the remainder of the specification, the AI models consist of advanced neural network architectures, including convolutional neural networks (CNN), deep neural networks (DNN), recurrent neural networks (RNN), deep residual convolutional neural networks, among others.

The self-regulation game module 204 determines a category associated with a self-regulation of the mental state status of the user. The category may indicate a zone of regulation for the student. In some implementations, the zone of regulation includes a blue zone, a green zone, a red zone, and a yellow zone.

The self-regulation game module 204 may obtain a second set of user data. The self-regulation game module 204 may input the second set of user data into the predictive model to determine a second score. The self-regulation game module 204 may compare the second score to the first score to determine feedback score. In some examples, if the second score is greater to or equal than the first score, the self-regulation game module 204 may retain the first gamification application in the category. In some examples, if the second score is less than the first score, The self-regulation game module 204 may remove the first gamification application from the category. The self-regulation game module 204 may determine a second gamification application.

The predictive system 104 shown in FIG. 2 includes user data 210. User data 210 may include survey data, cognitive data, creativity data, mindfulness data, or social behavior data. Other user data 210 may also include school reports, clinician reports, family reports, user reports, educator reports, and counsellor reports.

The AI-flagging module 206 determines, using the predictive AI model, a wellbeing index to identify at-risk user. In some embodiments, the system 700 sends, to a provider, a notification if the user is the at-risk user.

The predictive module 208 uses a predictive model such as a deep convolutional neural network model to predict patterns of psychological risks. For example, the predictive module can predict a student who is experiencing depression, anxiety, aggression, and ADHD.

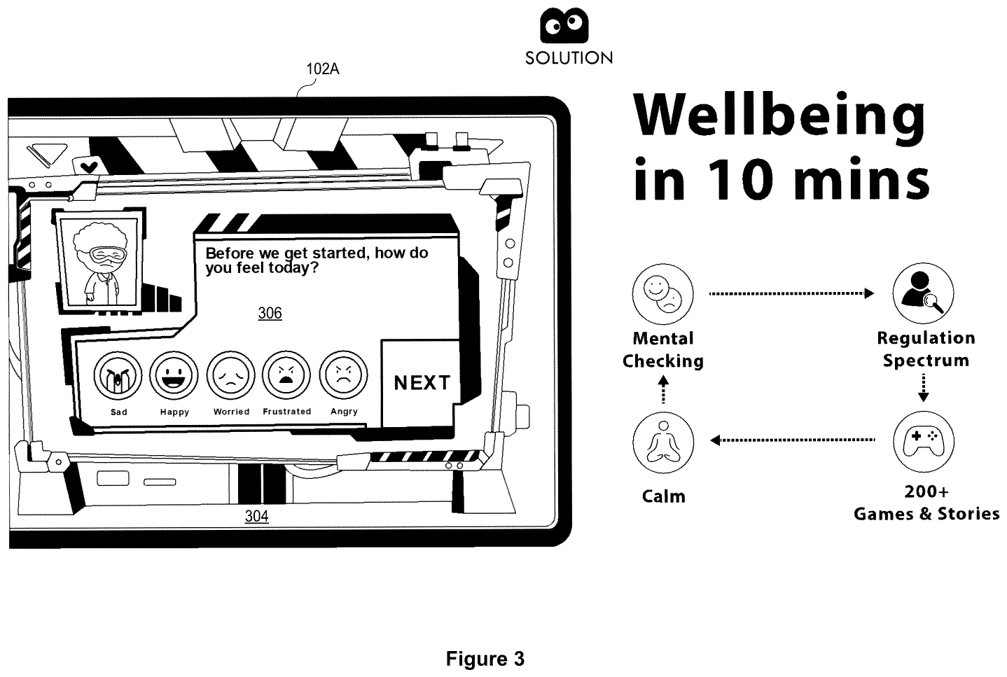

FIG. 3 is an example application that can be used to obtain a mental health status of a student according to one embodiment.

Referring to FIG. 3, the system includes a user device 102A on which a student an access an application 304 having an interface 306. The application 304 facilitates the collection of daily self-reported emotions from students through the interface 306. The emotions may include sad, happy, worried, frustrated, and angry. The student may select one of the emotions presented on the interface 306. Based on the daily self-reported emotions, the system determines the student's daily regulation score. The system may predict individualized games that help achieve regulation (coping).

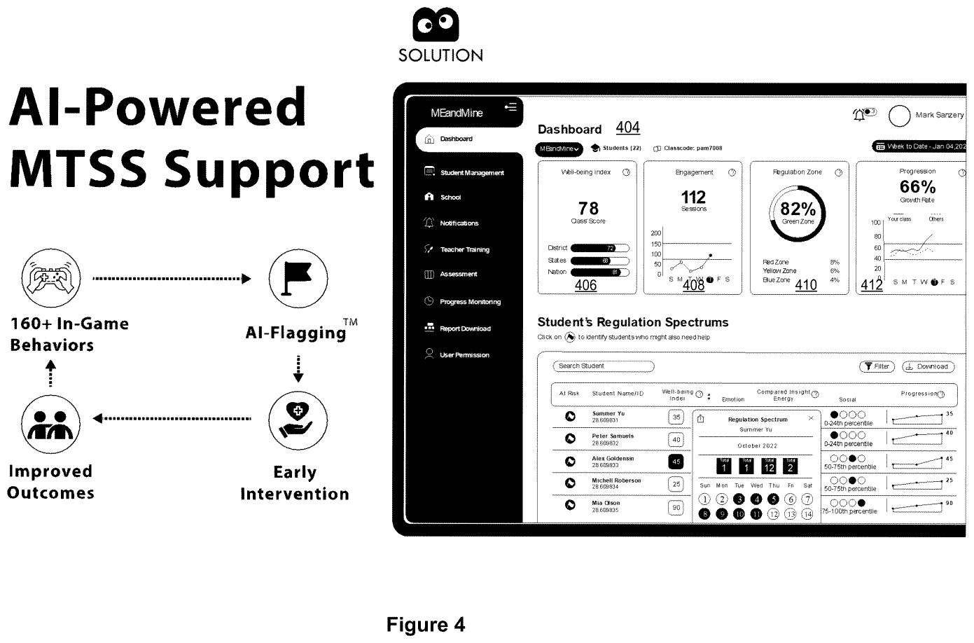

FIG. 4 is an example educator dashboard that can be used to monitor mental health status of a student according to one embodiment.

Referring to FIG. 4, the system also includes a user device 102B on which an educator can access an educator dashboard 404. Educator dashboard 404 is a centralized dashboard for educators, consolidating daily emotion, in-game behavior, and reward data. Educator dashboard 404 enables personalized student support with internal feedback and red flag labeling. Educator dashboard 404 provides ground-truth data from internal feedback and red-flagging enhances AI model learning. Educator dashboard 404 facilitates analysis across diverse groups for informed decision-making. Educator dashboard 404 displays AI-generated insights, empowering educators for holistic student well-being.

Is automatically synced. It might take up to 24 hours to reflect updates on your teacher's dashboard in the classroom tab, you can customize classroom settings to the needs of your classroom and individual students. Default daily session time is 15 minutes.

You can customize it to meet your needs. Students are set up to access all games, activities or stories. You can customize the access to better serve classroom routines. Now that your classroom is all set up and ready to go, I'll review some key features giving you insights into maximizing its capabilities.

Educator dashboard 404 may display a snapshot of the well-being index 406, engagement 408, regulation zone 410, and progression 412 for a specific time frame. The well-being index 406 reflects the class overall well-being by combining emotion, energy, and social scores. For example, a student who is in the green zone daily and socially engaged scores 100 percent. Well-being index 406 allows a comparison between a specific classroom score to those of school district, state, and nationwide. Progression 412 chart shows the growth of a classroom well-being over time. The regulation zone 410 displays the percentage of the students in the green zone and other zones.

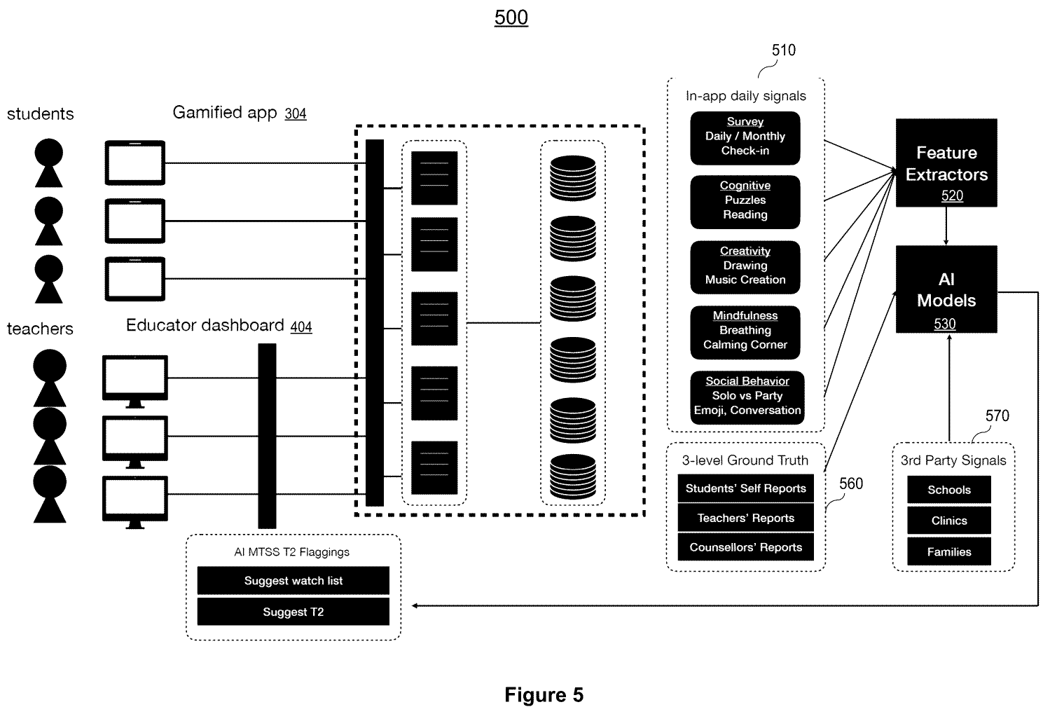

FIG. 5 is a block diagram illustrating an example of an architecture of a predictive system according to one embodiment.

Referring to FIG. 5, system 500 may obtain data from the gamified application 304 and the educator dashboard 404.

The system 500 may include a feature extractor 520 that identifies and extracts relevant features from the data 510. These features may be used to create a more informative dataset, which can be inputted into the predictive AI models 530 for the prediction.

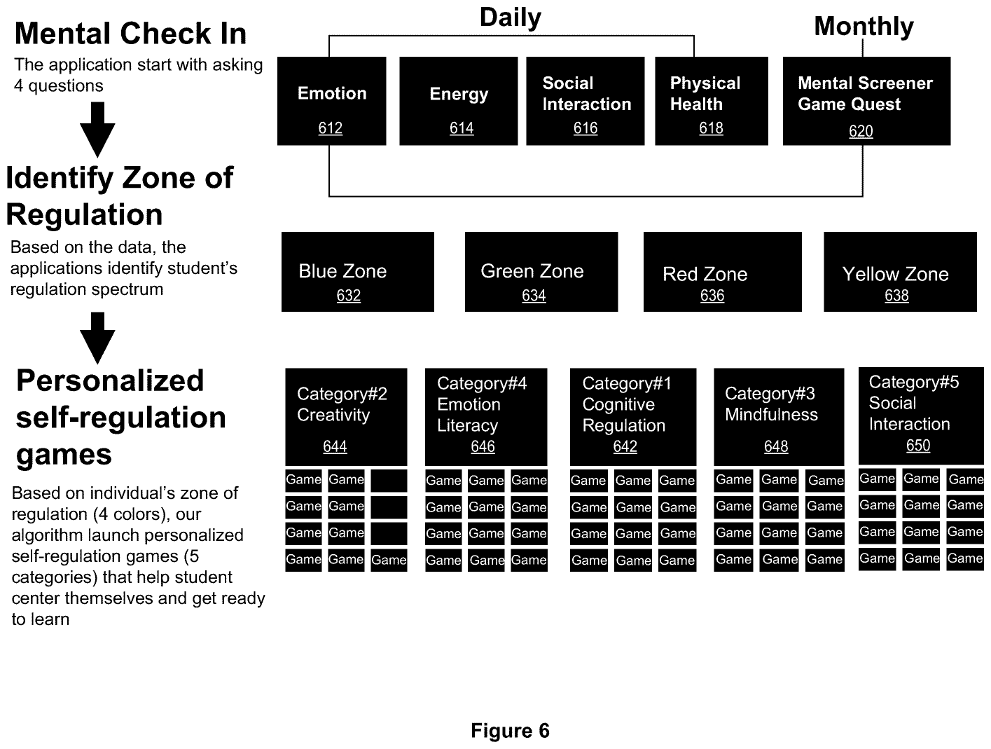

The data 510 may be obtained from the daily or monthly check-in which will be described in details in connection with FIG. 6. The data 510 may be obtained from the categories of games which will be described in details in connection with FIG. 6.

The system may also obtain additional data 560 including students' reports, teachers' reports, and counsellors' reports.

The system may also obtain additional data 570 including schools data, clinics data, and families data.

FIG. 6 is a block diagram illustrating an example of a technique of personalizing self-regulation games according to one embodiment.

Referring to FIG. 6, the system obtains, using measurement system, a first set of user data. In some embodiments, the system obtains a first set of data associated with a mental state of the student. For example, the first set of user data includes emotion 612, energy 614, social interaction 616, and physical health 618 of the user. The system may obtain the first set of user data daily. The system may also obtain additional data 620 including mental screener and game quest on a monthly basis. In some implementation, the system obtains the first set of user data via the application as described in connection with FIG. 3.

In some embodiments, the system inputs, the first set of user data into a predictive model to determine a first score.

In some embodiments, the system determines, using the score, an indicator associated with a mental state status of the user.

In some embodiments, the system determines, based on the indicator, a category associated with a self-regulation of the mental state status of the user.

The category may indicate a zone of regulation for the student. In some implementations, the zone of regulation includes a blue zone 632, a green zone 634, a red zone 636, and a yellow zone 638.

The blue zone 632 is when the students feel blue, down, and slow. In the blue zone, the students may feel sad, hurt, disappointed, bored.

The green zone 634 is when the students at their best. In this zone, the students are ready to learn they can play fairly. The students can even help out others. The green zone is associated with emotions such as joy, happiness, love, pride, because the students focus on their work satisfaction, on a job well done.

The red zone 636 is when the amygdala goes out of control, and it makes calming down difficult. Some emotions that may be associated with the red zone are angry, frustrated, anxious, and scared.

The yellow zone 638 is when the students are feeling a little uncomfortable and overwhelmed. Yellow means the students are overstimulated and unfocused. Some emotions that are associated with the yellow zone include excited, surprised, worried, impatient.

These zones help can identify a calming strategy to recenter the emotion of the students. For example, the system identifies an appropriate calming strategy for the students to return from the red zone to the green zone.

The calming strategy may include processing skills, distraction techniques, mindfulness techniques, and cognitive regulation games.

Processing skills may include reading, playing music or drawing. Distraction techniques may include fun tasks that help the students to relax their mind. Mindfulness techniques may include deep breathing. Cognitive regulation games may include gamification applications that trains the smart thinking brain.

The zone of regulation can help the students and others understand how the students are feeling. The zone of regulation can also identify the appropriate calming strategies to recenter the emotion of the students.

In some embodiments, the system determines a first gamification application (e.g., games) for the user based on the category.

Categories of games are designed and developed for self-regulation, designed to center from another colored zone back to the green zone which is the neurologically calm and in the state for learning.

Games that are associated with cognitive regulation 642 are suggested to help children in the red and yellow zones to get back to the calm state by redirecting them from emotional stimuli to logical thinking. These types of games are logic and strategy games, problem solving, reaction-time and focus games, spatial awareness and planning games. These hyper-casual, highly intellectual games provide children with a structured escape from intense emotional stimuli, engaging their prefrontal cortex and reinforcing calm, logical problem-solving.

Games that are associated with creativity 644 are suggested to support children in the blue zone, who may feel withdrawn and low-energy, it's essential to help them process and express their emotions in creative, non-verbal ways. Since verbal expression can be challenging in this state, engaging activities like creating music, crafting a face collage using food or objects, or drawing stories about their feelings offer valuable outlets for self-expression. These games not only allow children to document their mental and physical health but also provide psychologists with rich insights. Patterns within their creative work can serve as significant indicators, often signaling underlying anxiety or depression.

Games that are associated with emotion literacy 646 are suggested when children are in the green zone, they feel calm and focused, making it an ideal neurological state for learning. Games in this category are designed to enhance emotional literacy, including games that teach emotion vocabulary, help identify emotions through facial cues and physiological reactions, encourage understanding of emotional contexts through stories, assess the size of problems, and develop empathy. With this type of games, children can maximize their learning and deepen their knowledge of emotional awareness and understanding.

Games that are associated with mindfulness 648 are suggested to help children slow down their neurological responses, promoting a sense of calm and presence, especially when their energy levels are heightened in the red or yellow zones. Our interactive breathing games are volume-control, utilizing volume-detection capabilities to monitor children's inhaling and exhaling. When children breathe correctly, they receive real-time biofeedback rewards, reinforcing proper breathing techniques. This approach helps prevent common mistakes, such as rushing through breaths, which can lead to disturbed emotions and dizziness due to insufficient oxygen reaching the brain.

Games that are associated with social interaction 650 are suggested to students whose zone of regulation is in red, blue, yellow, green zones, recognizing that humans have an inherent need for social connection and belonging. Observing children's engagement, or lack thereof, in these interactions can serve as a significant indicator of social withdrawal, isolation, low self-esteem, or aggressive behavior.

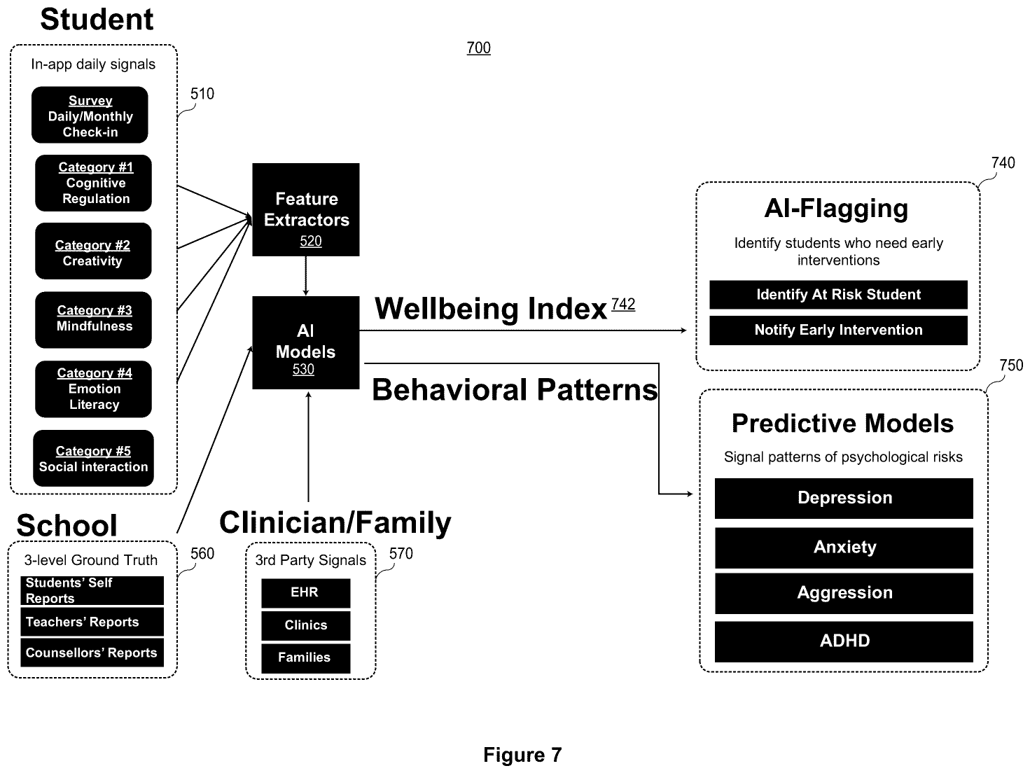

FIG. 7 is a block diagram illustrating an example of a predictive mental health flagging system 700 according to one embodiment. Elements 510, 520, 530, 560, and 570 have already been described above with respect to FIG. 5.

In some embodiments, the system obtains, using a first measurement system, a first data. For example, referring to FIG. 3, the system obtains user data using the application 304.

In some embodiments, the system inputs, the first data into a predictive model to determine a first score.

In some embodiments, the system determines, using the score, an indicator associated with a mental state status of the user.

In some embodiments, the system obtains, using a second measurement system, a second data.

In some embodiments, the system obtains, using a third measurement system, a third data.

Still referring to FIG. 7, the system 700 can provide AI-flagging 740 capability to identify students who need early interventions.

In some embodiments, the system 700 determines, using the predictive AI model 530, a wellbeing index 742 to identify at-risk user. In some embodiments, the system 700 sends, to a provider, a notification if the user is the at-risk user.

In some embodiments, the system 700 can provide a predictive model 750 capability to signal patterns of psychological risks. For example, the predictive model 750 can predict a student who is experiencing depression, anxiety, aggression, and ADHD.

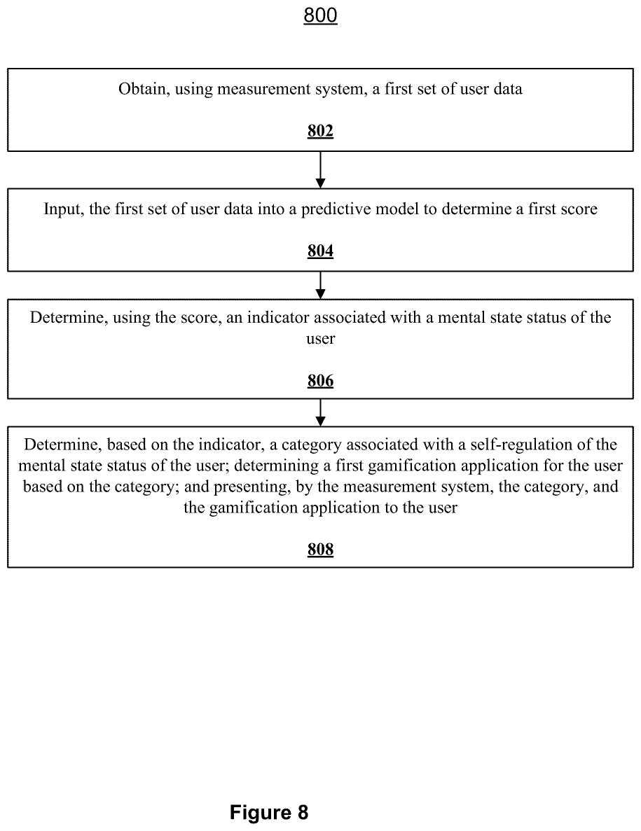

FIG. 8 is a flowchart of an example process 800. In some implementations, one or more process blocks of FIG. 8 may be performed by a device.

As shown in FIG. 8, process 800 may include obtaining, using measurement system, a first set of user data (block 802). For example, device may obtain, using measurement system, a first set of user data, as described above.

As also shown in FIG. 8, process 800 may include inputting, the first set of user data into a predictive model to determine a first score (block 804). For example, device may input, the first set of user data into a predictive model to determine a first score, as described above.

As further shown in FIG. 8, process 800 may include determining, using the score, an indicator associated with a mental state status of the user (block 806). For example, device may determine, using the score, an indicator associated with a mental state status of the user, as described above.

As also shown in FIG. 8, process 800 may include determining, based on the indicator, a category associated with a self-regulation of the mental state status of the user; determining a first gamification application for the user based on the category; and presenting, by the measurement system, the category, and the gamification application to the user (block 808). For example, device may determine, based on the indicator, a category associated with a self-regulation of the mental state status of the user; determining a first gamification application for the user based on the category; and presenting, by the measurement system, the category, and the gamification application to the user, as described above.

Process 800 may include additional implementations, such as any single implementation or any combination of implementations described below and/or in connection with one or more other processes described elsewhere herein. A first implementation, process 800 further includes obtaining a second set of user data; inputting the second set of user data into the predictive model to determine a second score; and comparing the second score to the first score to determine feedback score.

In a second implementation, alone or in combination with the first implementation, the second score is greater to or equal than the first score, further includes retaining the first gamification application in the category.

In a third implementation, alone or in combination with the first and second implementation, the second score is less than the first score, further includes removing the first gamification application from the category; and determining a second gamification application.

In a fourth implementation, alone or in combination with one or more of the first through third implementations, the first set of user data and the second set of user data include at least one of: survey data, cognitive data, creativity data, mindfulness data, or social behavior data.

In a fifth implementation, alone or in combination with one or more of the first through fourth implementations, further includes obtaining additional data including school reports, clinician reports, family reports, user reports, educator reports, and counsellor reports; and updating the predictive model based on the additional data.

In a sixth implementation, alone or in combination with one or more of the first through fifth implementations, the predictive model is a deep convolutional neural network model.

Although FIG. 8 shows example blocks of process 800, in some implementations, process 800 may include additional blocks, fewer blocks, different blocks, or differently arranged blocks than those depicted in FIG. 8. Additionally, or alternatively, two or more of the blocks of process 800 may be performed in parallel.

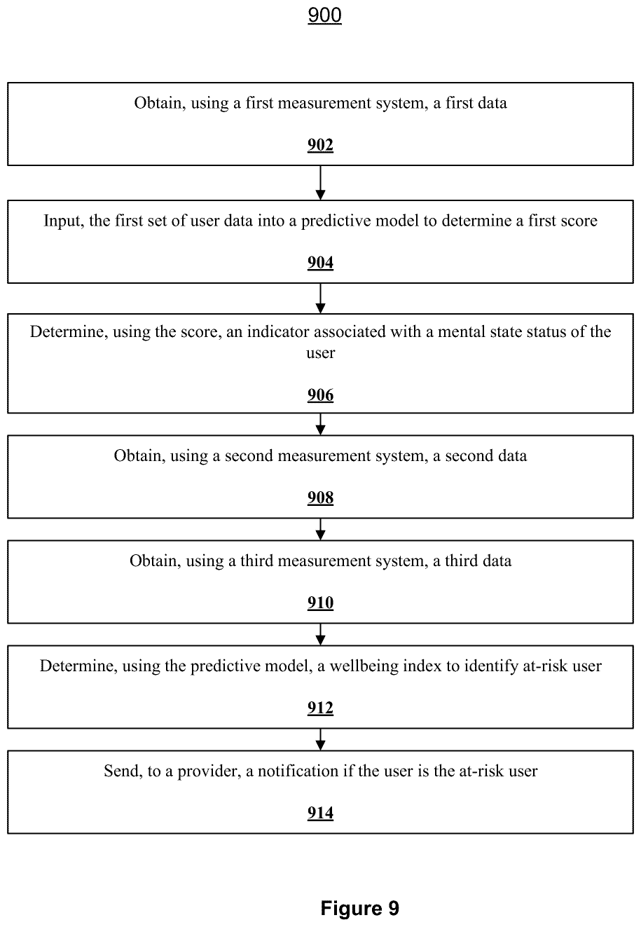

FIG. 9 is a flowchart of an example process 900. In some implementations, one or more process blocks of FIG. 9 may be performed by a device.

As shown in FIG. 9, process 900 may include obtaining, using a first measurement system, a first data (block 902). For example, device may obtain, using a first measurement system, a first data, as described above.

As also shown in FIG. 9, process 900 may include inputting, the first set of user data into a predictive model to determine a first score (block 904). For example, device may input, the first set of user data into a predictive model to determine a first score, as described above.

As further shown in FIG. 9, process 900 may include determining, using the score, an indicator associated with a mental state status of the user (block 906). For example, device may determine, using the score, an indicator associated with a mental state status of the user, as described above.

As also shown in FIG. 9, process 900 may include obtaining, using a second measurement system, a second data (block 908). For example, device may obtain, using a second measurement system, a second data, as described above.

As further shown in FIG. 9, process 900 may include obtaining, using a third measurement system, a third data (block 910). For example, device may obtain, using a third measurement system, a third data, as described above.

As also shown in FIG. 9, process 900 may include determining, using the predictive model, a wellbeing index to identify at-risk user (block 912). For example, device may determine, using the predictive model, a wellbeing index to identify at-risk user, as described above.

As further shown in FIG. 9, process 900 may include sending, to a provider, a notification if the user is the at-risk user (block 914). For example, device may send, to a provider, a notification if the user is the at-risk user, as described above.

Process 900 may include additional implementations, such as any single implementation or any combination of implementations described below and/or in connection with one or more other processes described elsewhere herein.

In a first implementation, the first data includes at least one: of survey data, cognitive data, creativity data, mindfulness data, or social behavior data.

In a second implementation, alone or in combination with the first implementation, the second data includes at least one of: school reports; clinician reports; or family reports.

In a third implementation, alone or in combination with the first and second implementation, the third data includes at least one of: user reports; educator reports; or counsellor reports.

In a fourth implementation, alone or in combination with one or more of the first through third implementations, the predictive model is a deep convolutional neural network model.

In a fifth implementation, alone or in combination with one or more of the first through fourth implementations, the first measurement system includes a user dashboard.

In a sixth implementation, alone or in combination with one or more of the first through fifth implementations, the second measurement system includes an educator dashboard.

In a seventh implementation, alone or in combination with one or more of the first through sixth implementations, the third measurement system includes a third party dashboard.

Although FIG. 9 shows example blocks of process 900, in some implementations, process 900 may include additional blocks, fewer blocks, different blocks, or differently arranged blocks than those depicted in FIG. 9. Additionally, or alternatively, two or more of the blocks of process 900 may be performed in parallel.

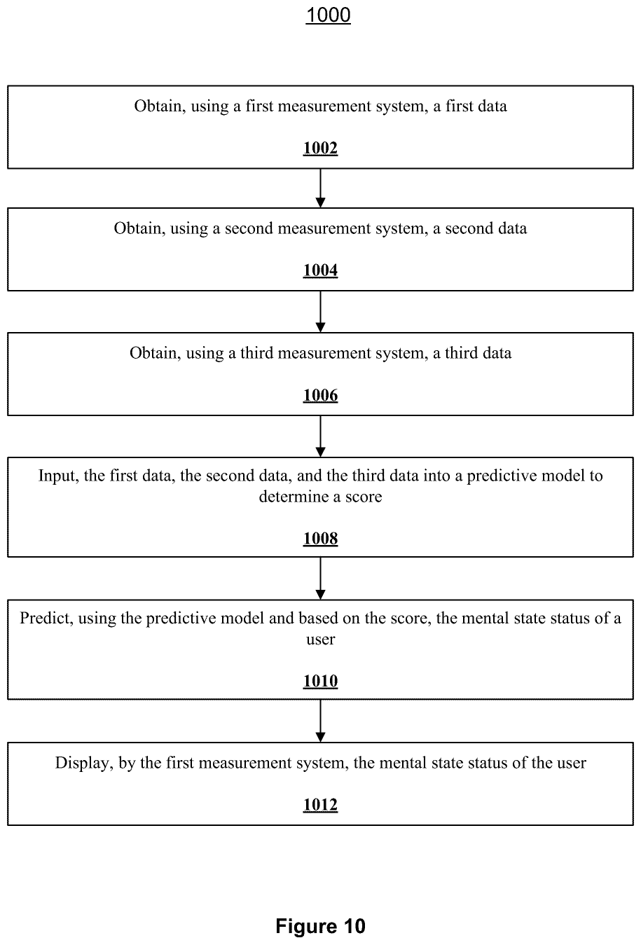

FIG. 10 is a flowchart of an example process 1000. In some implementations, one or more process blocks of FIG. 10 may be performed by a device.

As shown in FIG. 10, process 1000 may include obtaining, using a first measurement system, a first data (block 1002). For example, device may obtain, using a first measurement system, a first data, as described above.

As also shown in FIG. 10, process 1000 may include obtaining, using a second measurement system, a second data (block 1004). For example, device may obtain, using a second measurement system, a second data, as described above.

As further shown in FIG. 10, process 1000 may include obtaining, using a third measurement system, a third data (block 1006). For example, device may obtain, using a third measurement system, a third data, as described above.

As also shown in FIG. 10, process 1000 may include inputting, the first data, the second data, and the third data into a predictive model to determine a score (block 1008). For example, device may input, the first data, the second data, and the third data into a predictive model to determine a score, as described above.

As further shown in FIG. 10, process 1000 may include predicting, using the predictive model and based on the score, ta mental state status of an user (block 1010). For example, device may predict, using the predictive model and based on the score, a mental state status of a user, as described above.

As also shown in FIG. 10, process 1000 may include displaying, by the first measurement system, the mental state status of the user (block 1012). For example, device may display, by the first measurement system, the mental state status of the user, as described above.

Process 1000 may include additional implementations, such as any single implementation or any combination of implementations described below and/or in connection with one or more other processes described elsewhere herein. In a first implementation, the first data includes at least one: of survey data, cognitive data, creativity data, mindfulness data, or social behavior data.

In a second implementation, alone or in combination with the first implementation, the second data includes at least one of: school reports; clinician reports; or family reports.

In a third implementation, alone or in combination with the first and second implementation, the third data includes at least one of: user reports; educator reports; or counsellor reports.

In a fourth implementation, alone or in combination with one or more of the first through third implementations, the predictive model is a deep convolutional neural network model.

Although FIG. 10 shows example blocks of process 1000, in some implementations, process 1000 may include additional blocks, fewer blocks, different blocks, or differently arranged blocks than those depicted in FIG. 10. Additionally, or alternatively, two or more of the blocks of process 1000 may be performed in parallel.

Note that some or all of the components as shown and described above may be implemented in software, hardware, or a combination thereof. For example, such components can be implemented as software installed and stored in a persistent storage device, which can be loaded and executed in a memory by a processor (not shown) to carry out the processes or operations described throughout this application. Alternatively, such components can be implemented as executable code programmed or embedded into dedicated hardware such as an integrated circuit (e.g., an application specific IC or ASIC), a digital signal processor (DSP), or a field programmable gate array (FPGA), which can be accessed via a corresponding driver and/or operating system from an application. Furthermore, such components can be implemented as specific hardware logic in a processor or processor core as part of an instruction set accessible by a software component via one or more specific instructions.

Some portions of the preceding detailed descriptions have been presented in terms of algorithms and symbolic representations of operations on data bits within a computer memory. These algorithmic descriptions and representations are the ways used by those skilled in the data processing arts to most effectively convey the substance of their work to others skilled in the art. An algorithm is here, and generally, conceived to be a self-consistent sequence of operations leading to a desired result. The operations are those requiring physical manipulations of physical quantities.

It should be borne in mind, however, that all of these and similar terms are to be associated with the appropriate physical quantities and are merely convenient labels applied to these quantities. Unless specifically stated otherwise as apparent from the above discussion, it is appreciated that throughout the description, discussions utilizing terms such as those set forth in the claims below, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

Embodiments of the disclosure also relate to an apparatus for performing the operations herein. Such a computer program is stored in a non-transitory computer readable medium. A machine-readable medium includes any mechanism for storing information in a form readable by a machine (e.g., a computer). For example, a machine-readable (e.g., computer-readable) medium includes a machine (e.g., a computer) readable storage medium (e.g., read only memory (“ROM”), random access memory (“RAM”), magnetic disk storage media, optical storage media, flash memory devices).

The processes or methods depicted in the preceding figures may be performed by processing logic that comprises hardware (e.g., circuitry, dedicated logic, etc.), software (e.g., embodied on a non-transitory computer readable medium), or a combination of both. Although the processes or methods are described above in terms of some sequential operations, it should be appreciated that some of the operations described may be performed in a different order. Moreover, some operations may be performed in parallel rather than sequentially.

Embodiments of the present disclosure are not described with reference to any particular programming language. It will be appreciated that a variety of programming languages may be used to implement the teachings of embodiments of the disclosure as described herein.

In the foregoing specification, embodiments of the disclosure have been described with reference to specific exemplary embodiments thereof. It will be evident that various modifications may be made thereto without departing from the broader spirit and scope of the disclosure as set forth in the following claims. The specification and drawings are, accordingly, to be regarded in an illustrative sense rather than a restrictive sense.

What is claimed is:

1. A computer-implemented method, comprising:
obtaining, using a first measurement system, a first data;
inputting, the first data into a predictive model to determine a first score;
determining, using the score, an indicator associated with a mental state status of the user;
obtaining, using a second measurement system, a second data;
obtaining, using a third measurement system, a third data;
determining, using the predictive model, a wellbeing index to identify at-risk user; and
sending, to a provider, a notification if the user is the at-risk user.

2. The method of claim 1, wherein the first data includes at least one of:
survey data,
cognitive data,
creativity data,
mindfulness data, or
social behavior data.

3. The method of claim 1, wherein the second data includes at least one of:
school reports;
clinician reports; or
family reports.

4. The method of claim 1, wherein the third data includes at least one of:
user reports;
educator reports; or
counsellor reports.

5. The method of claim 1, wherein the predictive model is a deep convolutional neural network model.

6. The method of claim 1, wherein the first measurement system includes a user dashboard.

7. The method of claim 1, wherein the second measurement system includes an educator dashboard.

8. The method of claim 1, wherein the third measurement system includes a third party dashboard.

9. A non-transitory machine-readable medium having instructions stored therein, which when executed by a processor, cause the processor to perform operations, the operations comprising:
obtaining, using a first measurement system, a first data;
inputting, the first data into a predictive model to determine a first score;
determining, using the score, an indicator associated with a mental state status of the user;
obtaining, using a second measurement system, a second data;
obtaining, using a third measurement system, a third data;
determining, using the predictive model, a wellbeing index to identify at-risk user; and
sending, to a provider, a notification if the user is the at-risk user.

10. The non-transitory machine-readable medium of claim 9, wherein the first data includes at least one of:
survey data,
cognitive data,
creativity data,
mindfulness data, or
social behavior data.

11. The non-transitory machine-readable medium of claim 9, wherein the second data includes at least one of:
school reports;
clinician reports; or
family reports.

12. The non-transitory machine-readable medium of claim 9, wherein the third data includes at least one of:
user reports;
educator reports; or
counsellor reports.

13. The non-transitory machine-readable medium of claim 9, wherein the predictive model is a deep convolutional neural network model.

14. The non-transitory machine-readable medium of claim 9, wherein the first measurement system includes a user dashboard.

15. The non-transitory machine-readable medium of claim 9, wherein the second measurement system includes an educator dashboard.

16. The non-transitory machine-readable medium of claim 9, wherein the third measurement system includes a third party dashboard.

17. A system, comprising:
a processor; and
a memory coupled to the processor to store instructions, which when executed by the processor, cause the processor to perform operations, the operations comprising:
obtaining, using a first measurement system, a first data;
inputting, the first data into a predictive model to determine a first score;
determining, using the score, an indicator associated with a mental state status of the user;
obtaining, using a second measurement system, a second data;
obtaining, using a third measurement system, a third data;
determining, using the predictive model, a wellbeing index to identify at-risk user; and
sending, to a provider, a notification if the user is the at-risk user.

18. The system of claim 17, wherein the first data includes at least one of:
survey data,
cognitive data,
creativity data,
mindfulness data, or
social behavior data.

19. The system of claim 17, wherein the second data includes at least one of:
school reports;
clinician reports; or
family reports.

20. The system of claim 17, wherein the third data includes at least one of:
user reports;
educator reports; or
counsellor reports.
